(19) United States
(12) Patent Application Publication
(10) Pub. No.: US 2006/0159636 A1
(43) Pub. Date: Jul. 20, 2006

(54) Structurally modified titanium dioxides

(75) Inventors: Jurgen Meyer, Stockstadt (DE); Steffen Hasenzahl, Morris Plains, NJ (US); Heike Riedemann, Mombris (DE)

Correspondence Address: VENABLE LLP, P.O. BOX 34385, WASHINGTON, DC, 20045-9998, US

(73) Assignee: Degussa AG, Dusseldorf (DE)

(21) Appl. No.: 11/314,083

(22) Filed: Dec. 22, 2005

(30) Foreign Application Priority Data
Dec. 23, 2004 (EP) 04 030 585.6

(51) Int. Cl. A61K 8/29

(52) U.S. Cl. 424/59

(57) ABSTRACT

Pyrogenically prepared, structurally modified titanium dioxides or pyrogenically prepared, structurally modified titanium-dioxide mixed oxides are prepared by the titanium dioxides being treated with a ball mill and optionally being subsequently ground. They may be employed in sunscreen formulations.

The invention relates to pyrogenically prepared, structurally modified titanium dioxides or pyrogenically prepared, structurally modified titanium-dioxide mixed oxides, to a process for their preparation, and also to their use in sunscreen formulations.

It is known to employ pyrogenically prepared titanium dioxides and pyrogenically prepared titanium-dioxide/iron-oxide mixed oxides in sunscreen formulations (EP 0 609 533).

The known pyrogenically prepared titanium dioxides have the disadvantage that they and the sunscreen formulations produced therewith do not exhibit sufficient transparency when applied onto the skin.

Furthermore, an elaborate dispersal is necessary in the course of preparation.

A strong thickening effect in the course of dispersing, for example in cosmetic oils or water, renders difficult the production of dispersions or, to be more precise, sunscreen agents having a high content of titanium dioxide. The sunscreen agents that are produced with the known titanium dioxides give rise to a numb dermal sensation upon application.

The object of the invention is to prepare pyrogenically prepared titanium-dioxides that exhibit better transparency and also better dermal sensation in sunscreen agents.

The invention provides pyrogenically prepared, structurally modified titanium dioxides and also pyrogenically prepared, structurally modified titanium-dioxide mixed oxides.

The invention further provides a process for preparing the pyrogenically prepared, structurally modified titanium dioxides according to the invention and also the pyrogenically prepared, structurally modified titanium-dioxide mixed oxides according to the invention, said process being characterised in that the pyrogenically prepared titanium dioxide or pyrogenically prepared titanium-dioxide mixed oxide is treated by means of a ball mill and is optionally subsequently ground.

The pyrogenically prepared titanium dioxides that are listed in Table 1, for example, may be employed as educts or initial material.

TABLE 1

| | Aeroxide ® | Aeroxide ® | Aeroxide ® |
|---|---|---|---|
| | TiO₂ P25 | TiO₂ P25 S | TiO₂ PF2 |
| Appearance | white powder | white powder | white powder |
| Spec. surface area (BET)¹⁾ m²/g | 50 ± 15 | 50 ± 15 | 57.5 ± 12.5 |
| Mean size of primary particles nm | 21 | — | — |
| Tamped density (approx. value)²⁾ g/l | 130 | — | 80 |
| Bulk density (approx. value) g/l | — | 60-150 | — |
| Drying loss³⁾ (2 hrs. 105° C.) % | ≦1.5 | — | ≦2.0 |
| Annealing loss^{4) 7)} (2 hrs. 1000° C.) % | ≦2.0 | — | ≦3.0 |
| pH value⁵⁾ | 3.5-4.5 | — | 3.5-4.5 |
| As content ppm | — | ≦1.0 | — |
| Hg content ppm | — | ≦1.0 | — |
| Sb content ppm | — | ≦2.0 | — |
| Pb content ppm | — | ≦10 | — |
| SiO₂ content⁸⁾ wt. % | ≦0.200 | — | — |
| Al₂O₃ content⁸⁾ wt. % | ≦0.300 | — | — |
| Fe₂O₃ content⁸⁾ wt. % | ≦0.010 | — | 2.0-1 |
| TiO₂ content⁸⁾ wt. % | ≧99.5 | ≦99.0; ≦100.5 | ≧94.00 |
| HCl content¹⁰⁾ wt. % | ≦0.300 | ≦0.3 | ≦0.800 |
| Sieve residue⁶⁾ wt. % | ≦0.050 | — | — |
| (acc. to Mocker, 45 μm) | | | |

¹⁾Acc. to DIN 66131
²⁾Acc. to DIN EN ISO 787-11, JIS K 5101/20 (unsieved)
³⁾Acc. to DIN EN ISO 787-2, ASTM D280, JIS K 5101/23
⁴⁾Acc. to DIN EN ISO 3262-20, ASTM D 1208, JIS K 5101/24
⁵⁾Acc. to DIN EN ISO 787-9, ASTM D 1208, JIS K 5101/26
⁶⁾Acc. to DIN EN ISO 787-18, JIS K 5101/22
⁷⁾Based on dried substance (2 hrs. at 105° C.)
⁸⁾Annealed substance (2 hrs. at 1000° C.)
⁹⁾Special moisture-protecting packaging
¹⁰⁾HCl content is part of annealing loss

In particular, a titanium-dioxide powder containing iron oxide, which is known from document EP 0 609 533 A1, may be employed as educt.

The titanium-dioxide powder containing iron oxide may consist of a pyrogenically prepared iron-oxide/titanium-dioxide mixed oxide with a BET surface area from 10 m²/g to 150 m2/g which contains 0.5 wt. % to 50 wt. % iron oxide, relative to the total quantity, as a constituent of the mixed oxide.

Said powder may be prepared by anhydrous iron(III) chloride being vaporised, by being transported together with an inert gas, for example nitrogen, into the mixing chamber of a known burner, by being mixed there with hydrogen, air and gaseous titanium tetrachloride in a ratio that corresponds to the composition of the iron-oxide/titanium-dioxide mixed oxide, by the 4-component mixture undergoing combustion in a reaction chamber, and then by the solid iron-oxide/titanium-dioxide mixed oxide being separated from the gaseous reaction products and being optionally freed of adhering hydrogen chloride in humid air.

The pyrogenic preparation of titanium dioxide P 25 is known from Ullmann's Enzyklopädie der technischen Chemie, Volume 21, 4th Edition (1982). page 464.

Titanium dioxide P 25 is prepared by flame hydrolysis of titanium tetrachloride in accordance with the equation
TiCl₄+2 H₂+O₂→TiO₂+4 HCl.

Titanium dioxide PF 2, which is a titanium dioxide doped with 2% iron oxide, is likewise prepared by this process in accordance with the equations
TiCl₄+2 H₂+O₂→TiO₂+4 HCl
and
2 FeCl₃+3 H₂+1,5O₂→Fe₂O₃+6 HCl.

Both products, titanium dioxides PF 2 and P 25, consist crystallographically of approximately 80% anatase and 20% rutile. They exhibit a mean primary-particle size of about 20 nm.

The physiochemical properties of titanium dioxide PF 2 and P 25 are summarised in Table 1.

Titanium dioxide P 25 S exhibits the physicochemical properties listed in Table 1.

Furthermore, a pyrogenically prepared titanium dioxide according to DE 103 57 508.1 may be employed as educt which is present in aggregates of primary particles and which is characterised in that
it exhibits a BET surface area from 20 m²/g to 200 m²/g and
the half-width HW, in nanometres, of the primary-particle distribution exhibits values between
HW [nm]=a×BET^{f} with a=670×10⁹ m³/g and −1.3≦f≦−1.0, and
the proportion of particles having a diameter of more than 45 μm lies within a range from 0.0001 wt. % to 0.05 wt. %.

Said titanium dioxide can be prepared by
a titanium halide, preferably titanium tetrachloride, being vaporised at temperatures of less than 200° C., the vapours being transported into a mixing chamber by means of a carrying gas with a proportion of water vapour within a range from 1 g/m³ to 25 g/m³ carrying gas,
hydrogen, primary air, which may optionally be enriched with oxygen and/or preheated, and water vapour being transported separately into the mixing chamber,
whereby the proportion of water vapour lies within a range from 1 g/m³ to 25 g/m³ primary air,
the lambda value lies within the range from 1 to 9, and the gamma value lies within the range from 1 to 9,
   subsequently
the mixture—consisting of the vapour of the titanium halide, hydrogen, air and water vapour—being ignited in a burner, and the flame undergoing combustion into a reaction chamber sealed off from the ambient air, whereby
a vacuum from 1 mbar to 200 mbar is present in the reaction chamber, and the exit velocity of the reaction mixture out of the mixing chamber into the reaction space may lie within a range from 10 m/s to 80 m/s,
secondary air being introduced additionally into the reaction chamber, whereby
the ratio of primary air to secondary air may lie between 10 and 0.5,
the solids then being separated from gaseous substances and
the solids subsequently being treated with water vapour.

The structural modification may be carried out, for example, with a ball mill or with a ball mill operating continuously.

The after-grinding may be carried out, for example, by means of an air-jet mill, a toothed-disc mill or a pin-type mill.

The invention further provides the use of the pyrogenically prepared, structurally modified titanium dioxides according to the invention for the purpose of producing sunscreen agents or light-screening formulations or light-screening preparations.

The invention further provides sunscreen formulations which are characterised in that they contain pyrogenically prepared structurally modified titanium dioxides or pyrogenically prepared structurally modified titanium-dioxide mixed oxides.

The sunscreen formulations according to the present invention may preferably contain, besides one or more oleaginous phases, additionally one or more aqueous phases, and may be present, for example, in the form of W/O, O/W, W/O/W or O/W/O emulsions. Such formulations may preferably also be micro-emulsions, sticks, foams (so-called mousse), solid emulsions (i.e. emulsions that are stabilised by solids; e.g. Pickering emulsions), sprayable emulsions or hydrodispersions. Moreover, the preparations may advantageously also be oil-free and/or aqueous/alcoholic solutions.

According to the invention, (macroscopically) two-phase or multiphase systems are also advantageous. “Two-phase or multiphase” in the sense of the present invention means that two or more phases are present arranged separately in layers above one another. It is particularly advantageous in the sense of the present invention if at least one of the macroscopically visible phases constitutes a (W/O, O/W, micro-) emulsion. With this (macroscopic) way of looking at it, the emulsion is perceived as a phase, although to a person skilled in the art it is, of course, known that emulsions as such are formed from two or more phases which are homogenised with one another. The “emulsion phase” is stable in the long term, so that even over a relatively long period (months, years) no segregation or phase fractionation occurs within the emulsion.

The macroscopically visible phases or layers may advantageously be emulsified—for example by shaking—in a short time to form a homogeneous emulsion that is not stable in the long term but that segregates again over a period of minutes, hours or days to form two or more phases arranged in layers above one another.

It is particularly advantageous in the sense of the present invention if at least one of the macroscopically visible phases constitutes a microemulsion and at least one other of the macroscopically visible phases constitutes an oleaginous phase.

Particularly advantageous in the sense of the present invention are sprayable O/W emulsions, in particular O/W microemulsions.

The droplet diameters of the usual “simple”—that is to say, non-multiple—emulsions lie within the range from about 1 μm to about 50 μm. Such “macroemulsions” are, without further colouring additives, milky-white in colour and opaque. Finer “macroemulsions”, the droplet diameters of which lie within the range from about 0.5 μm to about 1 μm, are, again without colouring additives, bluish-white in colour and opaque. Such “macroemulsions” usually have a high viscosity.

On the other hand, the droplet diameter of microemulsions in the sense of the present invention lies within the range from approximately 50 nm to approximately 500 nm. Microemulsions of such a type are bluish-white to translucent in colour and mostly of low viscosity. The viscosity of many microemulsions of the O/W type is comparable to that of water.

An advantage of microemulsions is that active substances may be present in the disperse phase in substantially more finely dispersed manner than in the disperse phase of “macroemulsions”. A further advantage is that, by reason of their low viscosity, they can be atomized. If microemulsions are used as cosmetics, corresponding products are distinguished by a high degree of cosmetic elegance.

Advantageous in accordance with the invention are, in particular, O/W microemulsions that are obtainable with the aid of so-called phase-inversion-temperature technology and that contain at least one emulsifier (emulsifier A) which is chosen from the group of the emulsifiers having the following properties:
their lipophilia is dependent on the temperature in such a way that by increasing the temperature the lipophilia increases and by lowering the temperature the lipophilia of the emulsifier decreases.

Advantageous emulsifiers A are, for example, polyethoxylated fatty acids (PEG-100 stearate, PEG-20 stearate, PEG-150 laurath, PEG-8 distearate and suchlike) and/or polyethoxylated fatty alcohols (cetearath-12, cetearath-20, isoceteth-20, beheneth-20, laurath-9 etc.) and/or alkyl polyglycosides (cetearyl glycosides, stearyl glycosides, palmityl glycosides etc.).

To the extent that the phase inversion is initiated substantially by variation of the temperature, O/W emulsions, particularly O/W microemulsions, are obtainable, the size of the oil droplets being determined substantially by the concentration of the emulsifier(s) employed, in such a way that a higher emulsifier concentration brings about smaller droplets and a lower emulsifier concentration results in larger droplets. The sizes of the droplets are, as a rule, between 20 nm and 500 nm.

In the sense of the present invention it is optionally advantageous to use further W/O and/or O/W emulsifiers that are not covered by the definition of emulsifier A, for example in order to increase the water resistance of the preparations according to the present invention. Here use may be made, for example, of alkyl methicone copolyols and/or alkyl dimethicone copolyols (in particular, cetyl dimethicone copolyol, lauryl methicone copolyol), W/O emulsifiers (such as, for example, sorbitan stearate, glyceryl stearate, glycerol stearate, sorbitan oleate, lecithin, glyceryl isostearate, polyglyceryl-3-oleate, polyglyceryl-3-diisostearate, PEG-7 -hydrated castor oil, polyglyceryl-4-distearate, acrylate/C₁₀₋₃₀-alkyl-acrylate crosspolymer, sorbitan isostearate, poloxamer 101, polyglyceryl-2-dipolyhydroxystearate, polyglyceryl-3-diisostearate, polyglyceryl-4-dipolyhydroxystearate, PEG-30 dipolyhydroxystearate, diisostearoylpolyglyceryl-3-diisostearate, glycol distearate, polyglyceryl-3-dipolyhydroxystearate and/or fatty-acid esters of sulfuric or phosphoric acid (cetyl phosphate, trilaureth-4 phosphate, trioleth-8 phosphate, stearyl phosphate, cetearyl sulfate etc.).

Further advantageous sprayable O/W emulsions in the sense of the present invention are highly fluid cosmetic or dermatological hydrodispersions that contain at least one oleaginous phase and at least one aqueous phase, the preparation being stabilised by at least one gelling agent and not necessarily having to contain emulsifiers but possibly containing one or more emulsifiers.

Advantageous gelling agents for preparations of such a type are, for example, copolymers formed from C₁₀₋₃₀-alkyl acrylates and one or more monomers of acrylic acid, of methacrylic acid or esters thereof. The INCI name for such compounds is “acrylates/C₁₀₋₃₀ alkyl acrylate crosspolymer”. Advantageous, in particular, are the Pemulen® types TR1, TR2 and TRZ from Goodrich (Noveon).

Carbopols are also advantageous gelling agents for preparations of such a type. Carbopols are polymers of acrylic acid, in particular also acrylate/alkyl-acrylate copolymers. Advantageous Carbopols are, for example, types 907, 910, 934, 940, 941, 951, 954, 980, 981, 1342, 1382, 2984 and 5984, similarly the ETD types 2020, 2050 and Carbopol Ultrez 10. Further advantageous gelling agents for preparations of such a type are xanthan gum, cellulose derivatives and carob-bean flour.

Ethoxylated fatty alcohols or ethoxylated fatty acids (in particular, PEG-100 stearate, ceteareth-20) and/or other non-ionic surface-active substances may be used as possible (optional) emulsifiers.

Furthermore, the very highly fluid to sprayable emulsions may also advantageously be W/O or water-in-silicone-oil (WIS) emulsions. Advantageous, in particular, are W/O or W/S emulsions that contain
at least one silicon emulsifier (W/S) with an HLB value ≦8 and/or at least one W/O emulsifier with an HLB value ≦7 and
at least one O/W emulsifier with an HLB value >10.

Preparations of such a type further contain at least 20 wt. % lipids, it being possible for the lipid phase advantageously also to contain silicone oils or even to consist entirely of such oils. The silicone emulsifier(s) may advantageously be chosen from the group of the alkyl methicone copolyols and/or alkyl dimethicone copolyols (e.g. dimethicone copolyols, which are sold by Goldschmidt AG under the trade names ABIL® B 8842, ABIL® B 8843, ABIL® B 8847, ABIL® B 8851, ABIL® B 8852, ABIL® B 8863, ABIL® B 8873 and ABIL® B 88183, cetyl dimethicone copolyol [Goldschmidt AG/ABIL® EM 90], cyclomethicone dimethicone copolyol [Goldschmidt AG/ABIL® EM 97], lauryl methicone copolyol [Dow Corning Ltd. I Dow Corninge 5200 Formulation Aid], octyl dimethicone ethoxy glucoside [Wacker]).

The W/O emulsifier(s) with an HLB value <7 may advantageously be chosen from the following group: sorbitan stearate, sorbitan oleate, lecithin, glyceryl lanolate, lanolin, hydrated castor-oil, glyceryl isostearate, polyglyceryl-3-oleate, pentaerythrithol isostearate, methyl glucose dioleate, methyl glucose dioleate in a mixture with hydroxystearate and beeswax, PEG-7 hydrated castor oil, polyglyceryl-4-isostearate, hexyl laurate, acrylate/C₁₀₋₃₀-alkyl-acrylate crosspolymer, sorbitan isostearate, polyglyceryl-2-dipolyhydroxystearate, polyglyceryl-3-diisostearate, PEG-30 dipolyhydroxystearate, diisostearoyl-polyglyceryl-3-diisostearate, polyglyceryl-3-dipolyhydroxystearate, polyglyceryl-4-dipolyhydroxystearate, polyglyceryl-3-dioleate.

The O/W emulsifier(s) with an HLB value >10 may advantageously be chosen from the following group: glyceryl stearate in a mixture with ceteareth-20, ceteareth-25, ceteareth-6 in a mixture with stearyl alcohol, cetylstearyl alcohol in a mixture with PEG-40 castor oil and sodium cetylstearyl sulfate, triceteareth-4 phosphate, glyceryl stearate, sodium cetylstearyl sulfate, lecithin trilaureth-4 phosphate, laureth-4 phosphate, stearic acid, propylene glycol stearate SE, PEG-9 stearate, PEG-20 stearate, PEG-30 stearate, PEG-40 stearate, PEG-100 stearate, ceteth-2, ceteth-20, polysorbate-20, polysorbate-60, polysorbate-65, polysorbate-100, glyceryl stearate in a mixture with PEG-100 stearate, ceteareth-3, isostearyl glyceryl ether, cetylstearyl alcohol in a mixture with sodium cetylstearyl sulfate, PEG-40 stearate, glycol distearate, polyglyceryl-2-PEG-4 stearate, ceteareth-12, ceteareth-20, ceteareth-30, methyl glucose sesquistearate, steareth-10, PEG-20 stearate, steareth-21, steareth-20, isosteareth-20, PEG-45/dodecyl-glycol copolymer, glyceryl stearate SE, ceteth-20, PEG-20 methyl glucose sesquistearate, glyceryl stearate citrate, cetyl phosphate, cetearyl sulfate, sorbitan sesquioleate, triceteareth-4 phosphate, trilaureth-4 phosphate, polyglyceryl methyl glucose distearate, potassium cetylphosphate, isosteareth-10, polyglyceryl-2-sesquiisostearate, ceteth-10, isoceteth-20, glyceryl stearate in a mixture with ceteareth-20, ceteareth-12, cetylstearyl alcohol and cetyl palmitate, PEG-30 stearate, PEG-40 stearate, PEG-100 stearate.

Advantageous, furthermore, are aqueous alcoholic solutions. They may contain from 0 wt. % to 90 wt. % ethanol. Aqueous alcoholic solutions in the sense of the present invention may advantageously also contain solubilisers, such as, for example, PEG-40 or PEG-60 hydrogenated castor oil.

The preparations according to the present invention may advantageously also be used as cosmetic or dermatological impregnating solutions with which, in particular, water-insoluble substrates—such as, for example, woven or non-woven cloths—are moistened. Impregnating solutions of such a type are preferably highly fluid, in particular sprayable (such as, for example, PIT emulsions, hydrodispersions, W/O emulsions, oils, aqueous solutions etc.), and preferably have a viscosity of less than 2000 mPa.s, in particular less than 1500 mPa.s (measuring instrument: Haake Viskotester VT 02 at 25° C.). With their aid, cosmetic sunscreen cloths, skin-care cloths and suchlike, for example, are obtainable which represent the combination of a soft, water-insoluble material with the highly fluid cosmetic and dermatological impregnating solution.

The preparations according to the present invention may advantageously also be present in the form of anhydrous oils or oil gels or pastes. Advantageous oils are, for example, synthetic, semisynthetic or natural oils such as, for example, rapeseed oil, rice oil, avocado oil, olive oil, mineral oil, cocoglycerides, butylene glycol dicaprylate/dicaprate, C₁₂-C₁₅-alkyl benzoate, dicaprylyl carbonate, octyl dodecanol and suchlike. By way of oil-type gelling agents, use may be made of the most diverse waxes having a melting-point >25° C. Advantageous, furthermore, are gelling agents from the group of the aerosils, the alkyl galactomannans (e.g. N-Hance AG 200 and N-Hance AG 50 from Hercules) and polyethylene derivatives.

Particularly advantageous in the sense of the present invention are, furthermore, self-foaming, foam-like, after-foaming or foamable cosmetic and dermatological preparations.

The terms “self-foaming”, “foam-like”, “after-foaming” and “foamable” are to be understood to mean preparations from which foams—be it already during the production process, be it in the course of application by the consumer, or in some other way—can be produced in principle by intake of one or more gases. In foams of such a type the small gas bubbles are present (arbitrarily) distributed in one (or more) liquid phase(s), in which connection the (foamed-up) preparations do not necessarily have to have the appearance of a foam macroscopically. (Foamed-up) cosmetic or dermatological preparations according to the invention (hereinafter also designated as foams, for the sake of simplicity) may, for example, represent macroscopically visibly dispersed systems consisting of gases dispersed in liquids. But the foamy character may, for example, also become visible only under an (optical) microscope.

Moreover, foams according to the invention—in particular when the small gas bubbles are too small to be detected under an optical microscope—are also recognisable from the considerable increase in volume of the system.

It was particularly surprising, and is also based on an inventive step, that through the use of the alpha-olefin/maleic-anhydride copolymers according to the invention the intake of gases can be assisted and a stabilising and also distinctly foam-intensifying effect can be achieved also over a relatively long period of storage even at relatively high temperatures (e.g. 40° C.). In this connection, it was particularly astonishing that the use of special surfactants can be dispensed with. The intake of gases is, surprisingly, extraordonarily increased in comparison with the state of the art. For instance, a foam intensification with up to 100% increased gas volume can be achieved without using conventional foaming agents, such as surfactants, according to the state of the art.

It is possible by this means to generate formulations having a high gas volume (air and/or other gases such as oxygen, carbon dioxide, nitrogen, helium, argon, amongst others) in stable manner over a long period of storage at high temperatures.

The invention therefore further provides the use of one or more alpha-olefin/maleic-anhydride copolymers for the purpose of foam intensification of self-foaming, foam-like, after-foaming or foamable cosmetic and dermatological preparations.

The term “foam intensification” in the sense of the present invention is to be understood to mean that the intake of gases into the foams according to the invention is extraordinarily increased in comparison with the intake into otherwise identical preparations that contain no alpha-olefin/maleic-anhydride copolymers according to the invention. The foams according to the invention are accordingly able to absorb a distinctly higher volume of gas than preparations that contain no alpha-olefin/maleic-anhydride copolymers according to the invention.

The term “foam intensification” means, moreover, that the stability of the foamed-up preparations (the “foam stability”) is distinctly Improved in comparison with otherwise identical preparations that contain no alpha-olefin/maleic-anhydride copolymers according to the invention—i.e. through the use according to the invention a collapsing of the foams is temporally delayed.

Preparations of such a type in the sense of the present invention advantageously contain an emulsifier system that consists of
A) at least one emulsifier A chosen from the group of the wholly neutralised, partially neutralised or non-neutralised, branched and/or unbranched, saturated and/or unsaturated fatty acids with a chain length of 10 to 40 carbon atoms,
B) at least one emulsifier B chosen from the group of the polyethoxylated fatty-acid esters with a chain length of 10 to 40 carbon atoms and with a degree of ethoxylation from 5 to 100 and
C) at least one co-emulsifier C chosen from the group of the saturated and/or unsaturated, branched and/or unbranched fatty alcohols with a chain length of 10 to 40 carbon atoms.

The emulsifier(s) A is/are preferably chosen from the group of the fatty acids that are wholly or partially neutralised with conventional alkalis (such as, for example, sodium hydroxide and/or potassium hydroxide, sodium carbonate and/or potassium carbonate and also monoethanolamine and/or triethanolamine). Particularly advantageous are, for example, stearic acid and stearates, isostearic acid and isostearates, palmitic acid and palmitates and also myristic acid and myristates.

The emulsifer(s) B is/are preferably chosen from the following group: PEG-9 stearate, PEG-8 distearate, PEG-20 stearate, PEG-8 stearate, PEG-8 oleate, PEG-25 glyceryl trioleate, PEG-40 sorbitan lanolate, PEG-15 glyceryl ricinoleate, PEG-20 glyceryl stearate, PEG-20 glyceryl isostearate, PEG-20 glyceryl oleate, PEG-20 stearate, PEG-20 methyl glucose sesquistearate, PEG-30 glyceryl isostearate, PEG-20 glyceryl laurate, PEG-30 stearate, PEG-30 glyceryl stearate, PEG-40 stearate, PEG-30 glyceryl laurate, PEG-50 stearate, PEG-100 stearate, PEG-150 laurate. Particularly advantageous are, for example, polyethoxylated stearic acid esters.

According to the invention, the co-emulsifier(s) C is/are preferably chosen from the following group: behenyl alcohol (C₂₂H₄₅OH), cetearyl alcohol [a mixture of cetyl alcohol (C₁₆H₃₃OH) and stearyl alcohol (C₁₈H₃₇OH)], lanolin alcohols (wool-wax alcohols that represent the unsaponifiable alcohol fraction of wool wax, which is obtained after the saponification of wool wax). Particularly preferred are cetyl alcohol and cetylstearyl alcohol.

According to the invention, it is advantageous to choose the weight ratios of emulsifier A to emulsifier B to co-emulsifier C (A:B:C) as a:b:c, where a, b and c may represent, independently of one another, rational numbers from 1 to 5, preferably from 1 to 3. Particularly preferred is a weight ratio of approximately 1:1:1.

It is advantageous in the sense of the present invention to choose the total quantity of emulsifiers A and B and of co-emulsifier C from the range from 2 wt. % to 20 wt. %, advantageously from 5 wt. % to 15 wt. %, in particular from 7 wt. % to 13 wt. %, in each instance relative to the total weight of the formulation.

Particularly advantageous in the sense of the present invention are, furthermore, cosmetic or dermatological preparations that are stabilised only by extremely finely distributed solid particles. Such “emulsifier-free” emulsions are also designated as Pickering emulsions.

In Pickering emulsions an enrichment of the solid substance occurs at the oil/water phase boundary in the form of a layer, as a result of which a confluence of the disperse phases is prevented. Of essential importance in this connection are, in particular, the surface properties of the solid particles, which should display both hydrophilic and lipophilic properties.

The stabilising solid particles may also advantageously be subjected to water-repellent surface treatment (“coated”), in which connection an amphiphilic character of these solid particles is to be created or preserved. The surface treatment may consist in the solid particles being provided with a thin hydrophobic or hydrophilic layer by processes known as such.

The mean particle diameter of the microfine solid particles used as stabilisers is preferably chosen to be less than 100 μm, in particularly advantageous manner less than 50 μm. In this connection it is essentially irrelevant in which form (flakes, rodlets, globules etc.) or in which modification the solid particles that are used are present.

The microfine solid particles are preferably chosen from the group of the amphiphilic metal-oxide pigments. Advantageous are, in particular:
titanium dioxides (coated and uncoated): e.g. Eusolex T-2000 from Merck, titanium dioxide MT 100 Z from the Tayca Corporation
zinc oxides, e.g. Z-Cote and Z-Cote HP1 from BASF AG, MZ-300, MZ-500 and MZ-505M from the Tayca Corporation iron oxides.

Moreover, it is advantageous if the microfine solid particles are chosen from the following group: boron nitrides, starch derivatives (tapioca starch, sodium corn starch, octynyl succinate etc.), talcum, latex particles.

According to the invention, it is advantageous if the solids-stabilised emulsions contain distinctly less than 0.5 wt. % of one or more emulsifiers or are even entirely emulsifier-free.

Advantageous, moreover, in the sense of the invention are preparations in the form of sticks. Considered technically, most stick formulations are anhydrous fat mixtures of solid or semisolid waxes and liquid oils, with highly purified paraffin oils and waxes constituting the elementary matter of the stick.

Conventional base substances for stick-like preparations are, for example, liquid oils (such as, for example, paraffin oils, castor oil, isopropyl myristate, C₁₂₋₁₅-alkyl benzoate), semisolid constituents (e.g. Vaseline, lanolin), solid constituents (e.g. beeswax, ceresin and microcrystalline waxes or ozokerite) and/or high-melting waxes (e.g. carnauba wax, candelilla wax). Hydrous stick-like preparations are also known as such, it being possible for these also to be present in the form of W/O emulsions.

The cosmetic or dermatological light-screening formulations according to the invention may be composed as usual and may serve for the cosmetic or dermatological screening of light, furthermore for the treatment, care and cleansing of the skin and/or of the hair, and as make-up product in the field of decorative cosmetics.

In accordance with their structure, cosmetic or topical dermatological compositions in the sense of the present invention may, for example, be used as skin protection cream, cleansing milk, day cream or night cream etc. It is optionally possible and advantageous to use the compositions according to the invention as a base for pharmaceutical formulations.

With a view to use, the cosmetic and dermatological preparations are applied onto the skin and/or onto the hair in sufficient quantity in the manner that is conventional for cosmetics.

The cosmetic and dermatological preparations according to the invention may contain cosmetic auxiliary substances such as are used conventionally in such preparations, e.g. preserving agents, preserving aids, complexing agents, bactericides, perfumes, substances for preventing or intensifying foaming, dyestuffs, pigments that have a colouring effect, thickening agents, moistening and/or moist-keeping substances, fillers that improve the dermal sensation, fats, oils, waxes or other conventional constituents of a cosmetic or dermatological formulation, such as alcohols, polyols, polymers, foam stabilisers, electrolytes, organic solvents or silicone derivatives.

Advantageous preserving agents in the sense of the present invention are, for example, formaldehyde eliminators (such as, for example, DMDM hydantoin, which, for example, is available from Lonza under the trade name Glydant™), iodopropylbutyl carbamates (e.g. those available from Lonza under the trade names Glycacil-L, Glycacil-S, and/or Dekaben LMB from Jan Dekker), parabens (i.e. p-hydroxybenzoic acid alkyl esters, such as methyl, ethyl, propyl and/or butyl paraben), phenoxyethanol, ethanol, benzoic acid and suchlike. Ordinarily, the preserving system further also advantageously comprises, according to the invention, preserving aids such as, for example, octoxyglycerin, glycine soja etc.

Advantageous complexing agents in the sense of the present invention are, for example, EDTA, [S,S]-ethylenediamine disuccinate (EDDS), which, for example, is available from Octel under the trade name Octaquest, pentasodium ethylenediamine tetramethylene phosphonate, which, for example, is available from Monsanto under the trade name Dequest 2046, and/or iminodisuccinic acid, which is available, inter alia, from Bayer AG under the trade name Iminodisuccinat VP OC 370 (about 30% solution), and in solid form under the name Baypure CX 100.

Particularly advantageous preparations are obtained, furthermore, if antioxidants are employed as additive substances or active substances. According to the invention, the preparations advantageously contain one or ore antioxidants. By way of favourable antioxidants, which, however, are nonetheless to be used optionally, use may be made of all the antioxidants that are suitable or customary for cosmetic and/or dermatological applications.

Water-soluble antioxidants such as, for example, vitamins—for example, ascorbic acid and derivatives thereof—may be employed particularly advantageously in the sense of the present invention.

Preferred antioxidants are, furthermore, vitamin E and derivatives thereof and also vitamin A and derivatives thereof.

The quantity of the antioxidants (one or more compounds) in the preparations preferably amounts to 0.001 wt. % to 30 wt. %, particularly preferably 0.05 wt. % to 20 wt. %, in particular 0.1 wt. % to 10 wt. %, relative to the total weight of the preparation.

To the extent that vitamin E and/or derivatives thereof constitute the antioxidant(s), it is advantageous to choose the respective concentrations thereof from the range from 0.001 wt. % to 10 wt. %, relative to the total weight of the formulation.

To the extent that vitamin A or vitamin-A derivatives or carotenes or derivatives thereof constitute the antioxidant(s), it is advantageous to choose the respective concentrations thereof from the range from 0.001 wt. % to 10 wt. %, relative to the total weight of the formulation.

It is particularly advantageous if the cosmetic preparations according to the present invention contain cosmetic or dermatological active substances, preferred active substances being antioxidants which are able to protect the skin against oxidative exposure.

Further advantageous active substances In the sense of the present invention are natural active substances and/or derivatives thereof, such as, for example, alpha-liponic acid, phytoene, D-biotin, coenzyme Q10, alpha-glucosylrutin, carnitine, carnosine, natural and/or synthetic isoflavonoids, creatine, taurine and/or beta-alanine and also 8-hexadecene-1,16-dicarboxylic acid (dioic acid, CAS Number 20701-68-2; provisional INCI name octadecendioic acid).

Formulations according to the invention, which, for example, contain known anti-wrinkle active substances such as flavone glycosides (in particular alpha-glycosylrutin), co-enzyme Q10, vitamin E and/or derivatives and suchlike, are advantageously suitable, in particular, for the prophylaxis and treatment of cosmetic or dermatological changes to the skin such as occur, for example, in the course of ageing of the skin (such as, for example, dryness, roughness and formation of small wrinkles due to dryness, itching, diminished regreasing (e.g. after washing), visible vasodilations (cases of teleangiectasia, cuperosis), looseness and formation of (small) wrinkles, local hyperpigmentations, hypopigmentations and defective pigmentations (e.g. age spots), increased susceptibility to mechanical stress (e.g. cracking) and suchlike). Furthermore, they are advantageously suitable for combating the manifestation of dry or rough skin.

The aqueous phase of the preparations according to the present invention may advantageously contain conventional cosmetic auxiliary substances, such as, for example, alcohols, in particular those with a low C-number, preferably ethanol and/or isopropanol, diols or polyols with a low C-number and also ethers thereof, preferably propylene glycol, glycerin, butylene glycol, ethylene glycol, ethylene glycol monoethyl or monobutyl ether, propylene glycol monomethyl, monoethyl or monobutyl ether, diethylene glycol monomethyl or monoethyl ether and analogous products, polymers, foam stabilisers, electrolytes and also, in particular, one or more thickening agents, which may advantageously be chosen from the group comprising silicon dioxide, aluminium silicates, polysaccharides or derivatives thereof, e.g. hyaluronic acid, xanthan gum, hydroxypropylmethyl cellulose, particularly advantageously from the group of the polyacrylates, preferably a polyacrylate from the group of the so-called Carbopols [from Bf. Goodrich], for example Carbopols of the types 980, 981, 1382, 2984, 5984, ETD 2020, ETD 2050, Ultrez 10, in each instance individually or in combination.

The preparations according to the present invention may, furthermore, advantageously also contain self-tanning substances, such as, for example, dihydroxyacetone and/or melanin derivatives in concentrations from 1 wt. % up to 8 wt. %, relative to the total weight of the preparation.

Furthermore, the preparations according to the present invention may also advantageously contain repellents for protection against midges, ticks and spiders and suchlike. Advantageous are, for example, N,N-diethyl-3-methylbenzamide (trade name: Metadelphene, “DEET”), dimethyl phthalate (trade name: Palatinol M, DMP) and also, in particular, 3-(N-n-butyl-N-acetylamino)propionic acid ethyl ester (available from Merck under the trade name Insect Repellent™ 3535). The repellents may be employed both individually and in combination.

Designated as moisturisers are substances or substance mixtures that impart to cosmetic or dermatological preparations the property, after application or distribution on the surface of the skin, of reducing the release of moisture of the horny layer (also called transepidermal water loss (TEWL)) and/or of positively influencing the hydration of the horny layer.

Advantageous moisturisers in the sense of the present invention are, for example, glycerin, lactic acid and/or lactates, in particular sodium lactate, butylene glycol, propylene glycol, Biosaccaride Gum-1, glyucine soya, ethylhexyl oxyglycerin, pyrrolidonecarboxylic acid and urea. Furthermore, it is a particular advantage to use polymeric moisturisers from the group of the water-soluble polysaccharides and/or polysaccharides that are swellable in water and/or gellable with the aid of water.

Particularly advantageous are, for example, hyaluronic acid, chitosan and/or a fucose-rich polysaccharide that has been filed in the Chemical Abstracts under Registration Number 178463-23-5 and is available, for example, from SOLABIA S. A. under the name Fucogel™ 1000. Moisturisers may advantageously also be used as anti-wrinkle active substances for the prophylaxis and treatment of cosmetic or dermatological changes to the skin such as occur, for example, in the course of ageing of the skin.

The cosmetic or dermatological preparations according to the invention may, furthermore, advantageously—although not necessarily—contain fillers which, for example, further improve the sensory and cosmetic properties of the formulations and, for example, bring about or heighten a velvety or silky dermal sensation. Advantageous fillers in the sense of the present invention are starch and starch derivatives (such as, for example, tapioca starch, distarch phosphate, aluminium or sodium starch octenyl succinate and suchlike), pigments that have neither principally a UV-filter effect nor a colouring effect (such as, for example, boron nitride etc.) and/or Aerosil®.

The oleaginous phase of the formulations according to the invention is advantageously chosen from the group of the polar oils, for example from the group of the lecithins and the fatty-acid triglycerides, especially of the triglycerin esters of saturated and/or unsaturated, branched and/or unbranched alkanecarboxylic acids with a chain length from 8 to 24, in particular 12 to 18, C atoms. The fatty-acid triglycerides may, for example, advantageously be chosen from the group of the synthetic, semisynthetic and natural oils, such as, for example, cocoglyceride, olive oil, sunflower oil, soya oil, groundnut oil, rapeseed oil, almond oil, palm oil, coconut oil, castor oil, wheatgerm oil, grapeseed oil, thistle oil, evening-primrose oil, macadamia-nut oil and suchlike.

Advantageous furthermore in accordance with the invention are, for example, natural waxes of animal and vegetable origin, such as, for example, beeswax and other insect waxes and also berry wax, shea butter and/or lanolin (wool wax).

Further advantageous polar oil components may furthermore be chosen, in the sense of the present invention, from the group of the esters formed from saturated and/or unsaturated, branched and/or unbranched alkanecarboxylic acids with a chain length from 3 to 30 C atoms and saturated and/or unsaturated, branched and/or unbranched alcohols with a chain length from 3 to 30 C atoms and also from the group of the esters formed from aromatic carboxylic acids and saturated and/or unsaturated, branched and/or unbranched alcohols with a chain length from 3 to 30 C atoms. Such ester oils may then advantageously be chosen from the group comprising octyl palmitate, octyl cocoate, octyl isostearate, octyldodecyl myristate, octyl dodecanol, cetearyl isononanoate, isopropyl myristate, isopropyl palmitate, isopropyl stearate, isopropyl oleate, n-butyl -stearate, n-hexyl laurate, n-decyl -oleate, isooctyl stearate, isononyl stearate, isononyl isononanoate, 2-ethylhexyl palmitate, 2-ethylhexyl laurate, 2-hexyldecyl stearate, 2-octyldodecyl palmitate, stearyl heptanoate, oleyl oleate, oleyl erucate, erucyl oleate, erucyl erucate, tridecyl stearate, tridecyl trimellitate, and also synthetic, semisynthetic and natural mixtures of such esters, such as, for example, jojoba oil.

Furthermore, the oleaginous phase may advantageously be chosen from the group of the dialkyl ethers and dialkyl carbonates; advantageous are, for example, dicaprylyl ether (Cetiol OE) and/or dicaprylyl carbonate, for example that available from Cognis under the trade name Cetiol CC.

It is furthermore-preferred to choose the oil component(s) from the group comprising isoeicosane, neopentyl glycol diheptanoate, propylene glycol dicaprylate/dicaprate, caprylic/capric/diglyceryl succinate, butylene glycol dicaprylate/dicaprate, C₁₂₋₁₃-alkyl lactate, di-C₁₂₋₁₃-alkyl tartrate, triisostearine, dipentaerythritol hexacaprylate/hexacaprate, propylene glycol monoisostearate, tricapryline, dimethylisosorbide. It is advantageous, in particular, if the oleaginous phase of the formulations according to the invention exhibits a content of C₁₂₋₁₅-alkyl benzoate or consists completely of the latter.

Advantageous oil components are furthermore, for example, butyloctyl salicylate (for example, that available from CP Hall under the trade name Hallbrite BHB), hexadecyl benzoate and butyloctyl benzoate and mixtures thereof (Hallstar AB) and/or diethylhexyl naphthalate (Hallbrite TQ or Corapan TQ from H&R).

Arbitrary blends of such oil and wax components may also be employed advantageously in the sense of the present invention.

Furthermore, the oleaginous phase may also likewise advantageously contain non-polar oils, for example those which are chosen from the group of the branched and unbranched hydrocarbons and hydrocarbon waxes, in particular mineral oil, Vaseline (petrolatum), paraffin oil, squalane and squalene, polyolefins, hydrogenated polyisobutenes and isohexadecane. Among the polyolefins, polydecenes are the preferred substances.

The oleaginous phase may, furthermore, advantageously have a content of cyclic or linear silicone oils, or may consist completely of such oils, in which connection, however, it is preferred to use, besides the silicone oil or the silicone oils, an additional content of other oleaginous-phase components.

Silicone oils are high-molecular synthetic polymeric compounds in which silicon atoms are linked in the manner of a chain and/or lattice via oxygen atoms and the remaining valencies of the silicon are saturated by hydrocarbon residues (mostly methyl, more rarely ethyl, propyl, phenyl groups, inter alia). Systematically, the silicone oils are designated as polyorganosiloxanes. The methyl-substituted polyorganosiloxanes, which represent the quantitatively most significant compounds of this group and are distinguished by the following structural formula, 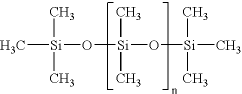
are also designated as polydimethylsiloxane or dimethicone (INCI). Dimethicones exist in various chain lengths and with various molecular weights.

Particularly advantageous polyorganosiloxanes in the sense of the present invention are, for example, dimethylpolysiloxanes [poly(dimethylsiloxane)], which are available, for example, from Th. Goldschmidt under the trade names Abil 10 to 10000. Advantageous, furthermore, are phenylmethylpolysiloxanes (INCI: phenyl dimethicones, phenyl trimethicones), cyclic silicones (octamethylcyclotetrasiloxane or decamethylcyclopentasiloxane), which according to INCI are also designated as cyclomethicones, amino-modified silicones (INCI: amodimethicones) and silicone waxes, e.g. polysiloxane-polyalkylene copolymers (INCI: stearyl dimethicone and cetyl dimethicones) and dialkoxydimethylpolysiloxanes (stearoxy dimethicone and behenoxy stearyl dimethicones), which are available from Th. Goldschmidt as various Abil-wax types. But other silicone oils may also be used advantageously in the sense of the present invention, for example cetyl dimethicone, hexamethylcyclotrisiloxane, polydimethylsiloxane, poly(methylphenylsiloxane).

The preparations according to the present invention may, furthermore, advantageously contain one or more substances from the following group of the siloxane elastomers, for example in order to increase the water resistance and/or the protection factor of the products:
a) siloxane elastomers that contain the units R₂SiO and RSiO_{1.5} and/or R₃SiO_{0.5} and/or SiO₂, where the individual residues R in each instance signify, independently of one another, hydrogen, C₁₋₂₄-alkyl (such as, for example, methyl, ethyl, propyl) or aryl (such as, for example, phenyl or tolyl), alkenyl (such as vinyl, for example), and the weight ratio of the units R₂SiO to RSiO_{1.5} is chosen from the range from 1:1 to 30:1;
b) siloxane elastomers that are insoluble in silicone oil and capable of swelling therein and that can be obtained by the addition reaction of an organopolysiloxane (1) which contains silicon-bound hydrogen with an organopolysiloxane (2) which contains unsaturated aliphatic groups, the quantitative proportions that are used being chosen in such a way that the quantity of hydrogen of organopolysiloxane (1) or of the unsaturated aliphatic groups of organopolysiloxane (2)
   lies within the range from 1 mol. % to 20 mol. % if the organopolysiloxane is not cyclic and
   lies within the range from 1 mol. % to 50 mol. % if the organopolysiloxane is cyclic.

In the sense of the present invention the siloxane elastomer(s) is/are advantageously present in the form of spherical powders or in the form of gels.

Siloxane elastomers that are present in the form of spherical powders and that are advantageous in accordance with the invention are the crosspolymers having the INCI name dimethicone/vinyl dimethicone, for example that which is available from DOW CORNING under the trade name DOW CORNING 9506 Powder.

It is particularly preferred if the siloxane elastomer is used in combination with oils from hydrocarbons of animal and/or vegetable origin, synthetic oils, synthetic esters, synthetic ethers or mixtures thereof.

It is quite particularly preferred if the siloxane elastomer is used in combination with unbranched silicone oils that are liquid or pasty at room temperature or with cyclic silicone oils or mixtures thereof. Particularly advantageous are organopolysiloxane elastomers having the INCI name dimethicone/polysilicone-11, quite particularly the Gransil types GCM, GCM-5, DMG-6, CSE gel, PM gel, LTX, ININ gel, AM-18 gel and/or DMCM-5 available from Grant Industries Inc.

It is quite extraordinarily preferred if the siloxane elastomer is used in the form of a gel consisting of siloxane elastomer and a lipid phase, the content of the siloxane elastomer in the gel amounting to 1 wt. % to 80 wt. %, preferably 10 wt. % to 60 wt. %, in each instance relative to the total weight of the gel.

It is advantageous in the sense of the present invention to choose the total quantity of the siloxane elastomers (active content) from the range from 0.01 wt. % to 10 wt. %, advantageously from 0.1 wt. % to 5 wt. %, in each instance relative to the total weight of the formulation. The cosmetic and dermatological preparations according to the invention may contain dyestuffs and/or coloured pigments, particularly if they are present in the form of decorative cosmetics. The dyestuffs and coloured pigments may be selected from the corresponding Positive List of the German Cosmetics Order [Positivliste der Kosmetikverordnung] or from the EC list of cosmetic colouring agents. In most cases they are identical with the dyestuffs that are licensed for foodstuffs. Advantageous coloured pigments are, for example, titanium dioxide, mica, iron oxides (e.g. Fe₂O₃, Fe₃O₄, FeO(OH)) and/or tin oxide. Advantageous dyestuffs are, for example, carmine, Prussian blue, chromium oxide green, ultramarine blue and/or manganese violet. It is particularly advantageous to choose the dyestuffs and/or coloured pigments from the Rowe Colour Index, 3rd Edition, Society of Dyers and Colourists, Bradford, England, 1971.

To the extent that the formulations according to the invention are present in the form of products that are used on the face, it is favourable to choose as dyestuff one or more substances from the following group: 2,4-dihydroxyazobenzene, 1-(2′-chloro-4′-nitro-1′-phenylazo)-2-hydroxynaphthaline, ceres red, 2-(sulfo-1-naphthylazo)-1-naphthol-4-sulfonic acid, calcium salt of 2-hydroxy-1,2′-azonaphthaline-1′-sulfo acid, calcium and barium salts of 1-(2-sulfo-4-methyl-1-phenylazo)-2-naphthylcarboxylic acid, calcium salt of 1-(2-sulfo-1-naphthylazo)-2-hydroxynaphthaline-3-carboxylic acid, aluminium salt of 1-(4-sulfo-1-phenylazo)-2-naphthol-6-sulfo acid, aluminium salt of 1-(4-sulfo-1-naphthylazo)-2-naphthol-3,6-disulfo acid, 1-(4-sulfo-1-naphthylazo)-2-naphthol-6,8-disulfo acid, aluminium salt of 4-(4-sulfo-1-phenylazo)-1-(4-sulfophenyl)-5-hydroxypyrazolone-3-carboxylic acid, aluminium and zirconium salts of 4,5-dibromofluorescein, aluminium and zirconium salts of 2,4,5,7-tetrabromofluorescein, 3′,4′,5′,6′-tetrachloro-2,4,5,7-tetrabromofluorescein and its aluminium salt, aluminium salt of 2,4,5,7-tetraiodofluorescein, aluminium salt of quinoophthalone disulfo acid, aluminium salt of indigo disulfo acid, red and black iron oxide (CIN: 77 491 (red) and 77 499 (black)), hydrated iron oxide (CIN: 77 492), manganese ammonium diphosphate and titanium dioxide.

Advantageous, furthermore, are oil-soluble natural dyestuffs such as, for example, paprika extracts, beta-carotene or cochineal.

Advantageous furthermore in the sense of the present invention are formulations having a content of pearlescent pigments. Preferred are, in particular, the types of pearlescent pigments listed in the following:
1. Natural pearlescent pigments, such as, for example,
   “pearl essence” (guanine/hypoxanthine mixed crystals derived from fish scales) and
“mother of pearl” (ground mussel shells)
2. Monocrystalline pearlescent pigments such as, for example, bismuth oxychloride (BiOCl)
3. Layer-substrate pigments: for example, mica/metal-oxide.

The base for pearlescent pigments is constituted, for example, by pulverulent pigments or castor-oil dispersions of bismuth oxychloride and/or titanium dioxide and also bismuth oxychloride and/or titanium dioxide on mica. Particularly advantageous is, for example, the lustrous pigment listed under CIN 77163.

Advantageous, furthermore, are, for example, the following pearlescent-pigment types based on mica/metal-oxide:

| Group | Coating/Layer-Thickness | Colour |
|---|---|---|
| Silver-white pearlescent | TiO₂: 40-60 nm | Silver |
| pigments | | |
| Interference pigments | TiO₂: 60-80 nm | Yellow |
| | TiO₂:80-100 nm | Red |
| | TiO₂:120-160 nm | Green |
| Coloured lustrous pigments | Fe₂O₃ | Bronze |
| | Fe₂O₃ | Copper |
| | Fe₂O₃ | Red |
| | Fe₂O₃ | Red-violet |
| | Fe₂O₃ | Red-green |
| | Fe₂O₃ | Black |
| Combination pigments | TiO₂/Fe₂O₃ | Gold tones |
| | TiO₂/Cr₂O₃ | Green |
| | TiO₂/Prussian blue | Deep blue |
| | TiO₂/Carmine | Red |

Particularly preferred are, for example, the pearlescent pigments available from Merck under the trade names Timiron, Colorona or Dichrona.

The list of the named pearlescent pigments is, of course, not intended to be limiting. Advantageous pearlescent pigments in the sense of the present invention are obtainable by numerous methods known as such. For example, substrates other than mica may also be coated with further metal oxides, such as, for example, silica and suchlike. Advantageous are, for example, SiO₂ particles coated with TiO₂ and Fe₂O₃ (“Ronaspheres”), which are marketed by Merck and which are particularly suitable for the optical reduction of fine wrinkles.

It may be an advantage, moreover, to dispense entirely with a substrate such as mica. Particularly preferred are iron pearlescent pigments that are prepared without the use of mica. Such pigments are available, for example, from BASF under the trade name Sicopearl Kupfer 1000.

Also particularly advantageous are, furthermore, effect pigments which are available in various colours (yellow, red, green, blue) from Flora Tech under the trade name Metasomes Standard/Glitter. The glitter particles here are present in mixtures with various auxiliary substances and dyestuffs (such as, for example, the dyestuffs having Colour Index (CI) Numbers 19140, 77007, 77289, 77491).

The dyestuffs and pigments may be present both individually and in a mixture and may also be mutually coated with one another, in which connection various colour effects are generally brought about by means of differing coating thicknesses. The total quantity of the dyestuffs and colour-imparting pigments is advantageously chosen from the range from, for example, 0.1 wt. % to 30 wt. %, preferably from 0.5 wt. % to 15 wt. %, in particular from 1.0 wt. % to 10 wt. %, in each instance relative to the total weight of the preparations.

It is also advantageous in the sense of the present invention to make available cosmetic and dermatological preparations, the principal purpose of which is not protection from sunlight but which nonetheless include a content of further UV-screening substances. For instance, UV-A or UV-B filter substances can usually be worked into day creams or make-up products. UV-screening substances, just like antioxidants and—if desired—preserving substances, also constitute an effective protection of the preparations themselves against deterioration. Favourable are, furthermore, cosmetic and dermatological preparations that are present in the form of a sunscreen agent.

Accordingly, the preparations in the sense of the present invention preferably contain at least one further UV-A, UV-B and/or wide-band filter substance. The formulations may optionally also contain, although not necessarily, one or more organic and/or inorganic pigments as UV filter substances, which may be present in the aqueous phase and/or in the oleaginous phase.

The preparations according to the present invention may, furthermore, advantageously also be present in the form of so-called oil-free cosmetic or dermatological emulsions which contain an aqueous phase and at least one UV filter substance that is liquid at room temperature by way of further phase and which, particularly advantageously, may also be free of further oleaginous components.

Particularly advantageous UV filter substances in the sense of the present invention that are liquid at room temperature are homomenthyl salicylate (INCI: homosalate), 2-ethylhexyl-2-cyano-3,3-diphenyl acrylate (INCI: octocrylene), 2-ethylhexyl-2-hydroxybenzoate (2-ethylhexyl salicylate, octyl salicylate, INCI: ethylhexyl salicylate) and esters of cinnamic acid, preferably 4-methoxycinnamic acid 2-ethylhexyl ester (2-ethylhexyl-4-methoxycinnamate, INCI: ethylhexyl methoxycinnamate) and 4-methoxycinnamic acid isopentyl ester (isopentyl-4-methoxycinnamate, INCI: isoamyl p-methoxycinnamate), 3-(4-(2,2-bis-ethoxycarbonylvinyl)phenoxy)propenyl)methoxysiloxane/dimethylsiloxane copolymer which, for example, is available from Hoffmann La Roche under the trade name Parsol® SLX.

Preferred inorganic pigments are metal oxides and/or other metal compounds that are sparingly soluble or insoluble in water, particularly oxides of titanium (TiO₂), of zinc (ZnO), of iron (e.g. Fe₂O₃), of zirconium (ZrO₂), of silicon (SiO₂), of manganese (e.g. MnO), of aluminium (Al₂O₃), of cerium (e.g. Ce₂O₃), mixed oxides of the corresponding metals and also blends of such oxides, and also the sulfate of barium (BaSO₄).

In the sense of the present invention the pigments may also advantageously find application in the form of commercially available oleaginous or aqueous predispersions. Dispersing agents and/or solubilisation promoters may advantageously be added to these predispersions.

According to the invention, the pigments may advantageously be surface-coated, in which connection a hydrophilic, amphiphilic or hydrophobic character, for example, is intended to be created or preserved. This surface treatment may consist in the pigments being provided with a thin hydrophilic and/or hydrophobic inorganic and/or organic layer by processes known as such. The various surface coatings in the sense of the present invention may also contain water.

Inorganic surface coatings in the sense of the present invention may consist of aluminium oxide (Al₂O₃), aluminium hydroxide Al(OH)₃, or hydrated aluminium oxide (also: alumina, CAS No.: 1333-84-2), sodium hexametaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may be found on their own, in combination, and/or in combination with organic coating materials.

Organic surface coatings in the sense of the present invention may consist of vegetable or animal aluminium stearate, vegetable or animal stearic acid, lauric acid, dimethylpolysiloxane (also: dimethicone), methylpolysiloxane (methicone), simethicone (a mixture of dimethylpolysiloxane with an average chain length from 200 to 350 dimethylsiloxane units and silica gel) or alginic acid. These organic surface coatings may be found on their own, in combination, and/or in combination with inorganic coating materials. Zinc-oxide particles and predispersions of zinc-oxide particles that are suitable in accordance with the invention are available from the specified companies under the following trade names:

| Trade Name | Coating | Manufacturer |
|---|---|---|
| Z-Cote HP 1 | 2% dimethicone | BASF |
| Z-Cote | / | BASF |
| ZnO NDM | 5% dimethicone | H & R |
| MZ-303S | 3% methicone | Tayca Corporation |
| MZ-505S | 5% methicone | Tayca Corporation |

Suitable titanium-dioxide particles and predispersions of titanium-dioxide particles are available from the specified companies under the following trade names:

| Trade Name | Coating | Manufacturer |
|---|---|---|
| MT-100TV | aluminium hydroxide/ | Tayca Corporation |
| | stearic acid | |
| MT-100Z | aluminium hydroxide/ | Tayca Corporation |
| | stearic acid | |
| Eusolex T-2000 | alumina/ | Merck KGaA |
| | simethicone | |
| titanium dioxide | Octyltrimethoxy | Degussa |
| T805 (Uvinul TiO₂) | silane | |
| Tioveil AQ 10PG | alumina/silica | Solaveil/Uniquema |
| Eurolex T-aqua | water/alumina/ | Merck |
| | sodium | |
| | metaphosphate | |

Other advantageous pigments are latex particles. Latex particles that are advantageous in accordance with the invention are those described in the following documents: U.S. Pat. No. 5,663,213 and EP 0 761 201. Particularly advantageous latex particles are those which are formed from water and styrene/acrylate copolymers and which are available, for example, from Rohm & Haas under the trade name “Alliance SunSphere”.

Advantageous UV-A filter substances in the sense of the present invention are dibenzoylmethane derivatives, in particular 4-(tert.-butyl)-4′-methoxydibenzoylmethane (CAS No. 70356-09-1) which is sold by Givaudan under the brand Parsol™ 1789 and sold by Merck under the trade name Eusolex™ 9020.

Advantageous further UV filter substances in the sense of the present invention are sulfonated, water-soluble UV filters, such as, for example:
phenylene-1,4-bis-(2-benzimidazyl)-3,3′-5,5′-tetrasulfonic acid and its salts, especially the corresponding sodium, potassium or triethanolammonium salts, in particular phenylene-1,4-bis-(2-benzimidazyl)-3,3′-5,5′-tetrasulfonic acid-bis-sodium salt having the INCI name disodium phenyl dibenzimidazole tetrasulfonate (CAS No.: 180898-37-7) which is available, for example, from Haarmann & Reimer under the trade name Neo Heliopan AP;
salts of 2-phenylbenzimidazole-5-sulfonic acid, such as its sodium, potassium or triethanolammonium salt and also the sulfonic acid itself having the INCI name phenylbenzimidazole sulfonic acid (CAS No. 27503-81-7), which is available, for example, from Merck under the trade name Eusolex 232 or from Haarmann & Reimer under the trade name Neo Heliopan Hydro;
1,4-di(2-oxo-10-sulfo-3-bornylidenemethyl)benzene (also: 3,3′-(1,4-phenylenedimethylene)-bis-(7,7-dimethyl-2-oxo-bicyclo-[2.2.1]hept-1-ylmethanesulfonic acid) and salts thereof (especially the corresponding 10-sulfato compounds, in particular the corresponding sodium, potassium or triethanolammonium salt), which is also designated as benzene-1,4-di(2-oxo-3-bornylidenemethyl-10-sulfonic acid). Benzene-1,4-di(2-oxo-3-bornylidenemethyl-10-sulfonic acid) has the INCI name terephtalidene dicamphor sulfonic acid (CAS No.: 90457-82-2) and is available, for example, from Chimex under the trade name Mexoryl SX;
Sulfonic-acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

Advantageous UV filter substances in the sense of the present invention are, furthermore, benzoxazole derivatives that are distinguished by the following structural formula 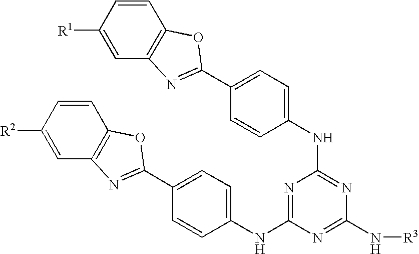
in which R¹, R² and R³ are chosen, independently of one another, from the group of the branched or unbranched, saturated or unsaturated alkyl residues with 1 to 10 carbon atoms. According to the invention, it is particularly advantageous to choose the residues R¹ and R² to be the same, in particular from the group of the branched alkyl residues with 3 to 5 carbon atoms. Furthermore, it is particularly advantageous in the sense of the present invention if R³ represents an unbranched or branched alkyl residue with 8 carbon atoms, in particular the 2-ethylhexyl residue.

A benzoxazole derivative that is particularly preferred according to the invention is 2,4-bis-[5-1(dimethylpropyl)benzoxazole-2-yl-(4-phenyl)-imino]-6-(2-ethylhexyl)-imino-1,3,5-triazine having the CAS No. 288254-16-0, which is distinguished by the structural formula 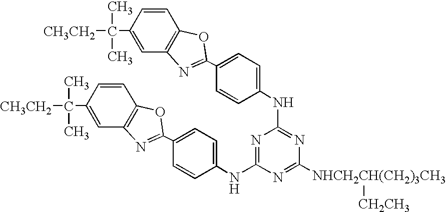
and is available from 3V Sigma under the trade name Uvasorb TM K2A.

The benzoxazole derivative(s) is/are advantageously present in dissolved form in the cosmetic preparations according to the invention. But it may optionally also be an advantage if the benzoxazole derivative(s) is/are present in pigmentary—i.e. undissolved—form, for example in particle sizes from 10 nm to 300 nm.

Advantageous UV filter substances in the sense of the present invention are, furthermore, so-called hydroxybenzophenones. Hydroxybenzophenones are distinguished by the following structural formula: 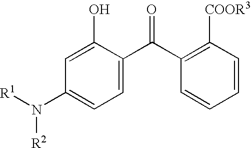
in which
R¹ and R² signify, independently of one another, hydrogen, C₁-C₂₀-alkyl, C₃-C₁₀-cycloalkyl or C₃-C₁₀-cycloalkenyl, where the substituents R¹ and R² may form, together with the nitrogen atom to which they are bonded, a 5-membered or 6-membered ring and
R³ signifies a C₁-C₂₀-alkyl residue.

A particularly advantageous hydroxybenzophenone in the sense of the present invention is 2-(4′-diethylamino-2′-hydroxybenzoyl)benzoic acid hexylester (also: aminobenzophenone), which is distinguished by the following structure: 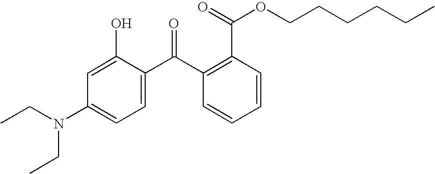
and is available from BASF under the name Uvinul A Plus.

Advantageous UV filter substances in the sense of the present invention are, furthermore, so-called wide-band filters, i.e. filter substances that absorb both UV-A and UV-B radiation.

Advantageous wide-band filters or UV-B filter substances are, for example, triazine derivatives, such as, for example,
2,4-bis-[4-(2-ethyl-hexyloxy)-2-hydroxy]phenyl-6-(4-methoxyphenyl)-1,3,5-triazine (INCI: bis-ethylhexyloxylphenol methoxyphenyl triazine), which is available from CIBA-Chemikalien GmbH under the trade name Tinosorb™ S;
dioctylbutylamidotriazone (INCI: diethylhexyl butamido triazone), which is available from Sigma 3 V under the trade name UVASORB HEB;
4,4′,4″-(1,3,5-triazine-2,4,6-triyltramino)-tris-benzoic acid-tris-(2-ethylhexyl ester), also: 2,4,6-tris-[anilino-(p-carbo-2′-ethyl-1′-hexyloxy)]-1,3,5-triazine (INCI: ethylhexyl triazone), which is marketed by BASF Aktiengesellschaft under the trade name UVINUL™ T 150;
2-[4,6-bis(2,4-dimethylphenyl)-1,3,5-triazine-2-yl]-5-(octyloxy)phenol (CAS No.: 2725-22-6).

An advantageous wide-band filter in the sense of the present invention is also 2,2-methylene-bis-(6-(2H-benzotriazole-2-yl)-4-(1,1,3,3-tetramethylbutyl)phenol) (INCI: methylene bis-benztriazolyl tetramethylbutylphenol), which is available, for example, from CIBA-Chemikalien GmbH under the trade name Tinosorb™ M.

An advantageous wide-band filter in the sense of the present invention is, furthermore, 2-(2H-benzotriazole-2-yl)-4-methyl-6-[2-methyl-3-[1,3,3,3-tetramethyl-1-[(trimethylsilyl)oxy]disiloxanyl]propyl]phenol (CAS No.: 155633-54-8) having the INCI name drometrizole trisiloxane.

The further UV filter substances may be oil-soluble or water-soluble. Advantageous oil-soluble filter substances are, for example:
3-benzylidenecamphor derivatives, preferably 3-(4-methylbenzylidene)camphor, 3-benzylidenecamphor;
4-aminobenzoic acid derivatives, preferably 4-dimethylaminobenzoic acid 2-ethylhexyl ester, 4-dimethylaminobenzoic acid amyl ester;
2,4,6-trianilino-(p-carbo-2′-ethyl-1′-hexyloxy)-1,3,5-triazine;
esters of benzalmalonic acid, preferably 4-methoxybenzalmalonic acid di(2-ethylhexyl) ester;
esters of cinnamic acid, preferably 4-methoxycinnamic acid 2-ethylhexyl ester, 4-methoxycinnamic acid isopentyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone and also
UV filters bound to polymers.

Advantageous water-soluble filter substances are, for example: sulfonic-acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

A further light-screening filter substance to be used advantageously in accordance with the invention is ethylhexyl-2-cyano-3,3-diphenyl acrylate (octocrylene), which is available from BASF under the name Uvinul® N 539 T.

Particularly advantageous preparations in the sense of the present invention, which are distinguished by high or very high UV-A protection, preferably further contain, in addition to the filter substance(s) according to the invention, other UV-A filters and/or wide-band filters, in particular dibenzoylmethane derivatives [for example, 4-(tert.-butyl)-4′-methoxydibenzoylmethane] and/or 2,4-bis-[4-(2-ethylhexyloxy)-2-hydroxy]phenyl-6-(4-methoxyphenyl)-1,3,5-triazine and/or phenylene-1,4-bis-(2-benzimidazyl)-3,3′-5,5′-tetrasulfonic acid-bis-sodium salt, in each instance individually or in arbitrary combinations with one another.

The list of the named UV filters that can be employed in the sense of the present invention is, of course, not intended to be limiting.

The preparations according to the present invention advantageously contain the substances that absorb UV radiation in the UV-A and/or UV-B ranges in a total quantity from, for example, 0.1 wt. % to 30 wt. %, preferably 0.5 wt. % to 20 wt. %, in particular 1.0 wt. % to 15.0 wt. %, in each instance relative to the total weight of the preparations, in order to make cosmetic preparations available that protect the hair or the skin from the entire range of ultraviolet radiation.

The preparations in the sense of the present invention may, furthermore, advantageously contain other substances that increase the water resistance of the products.

Advantageous are, for example, polyoxyethylene-polyoxypropylene block polymers that are water-soluble or dispersible in water (CTFA name: polaxamers, CAS No. 9003-11-6) having the following structure: 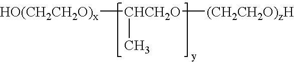
where x, y and z represent integers from the range from 2 to 130, in particular from 15 to 100, and x and z are equal but are chosen independently of y.

To be used advantageously amongst these are, in particular, polaxamer 188 [with x=75, y=30 and z=75], which can be obtained from BASF under the trade name Lutrol F 68 (formerly: Pluronic F 68), polaxamer 185 [with x=19, y=30 and z=19] (Lubrajel WA from ISP), polaxamer 235 [with x=27, y=39 and z=27] (Pluronic F 85 from BASF) and/or polaxamer 238 [with x=97, y=39 and z=97] (Pluronic F 88 from BASF).

Further advantageous substances, which may contribute to the heightening of the water resistance but which are worked into the oleaginous phase of the preparations according to the present invention, are certain wax components, such as acetylated glycol stearate with tristearine (e.g. Unitwix from ISP having the INCI: acetylated glycol stearate and tristearine), C₁₈₋₃₆ fatty-acid triglyceride (e.g. Syncrowax HGLC from Crode GmbH having the INCI: C₁₈₋₃₆ acid triglyceride) and furthermore the substance available from New Phase Technologies under the trade name “Perfroma V 825” (synthetic wax) and also PEG-45 dodecyl glycol copolymer (INCI: PEG-45 dodecyl glycol copolymer), PEG-22 dodecyl glycol copolymer (INCI: PEG-22 dodecyl glycol copolymer), methoxy PEG-22 dodecyl glycol copolymer (INCI: methoxy PEG-22 dodecyl glycol copolymer), which are available from AKZO Nobel.

It is particularly advantageous in the sense of the present invention to combine the polymers that are used in accordance with the invention with one or more of the named substances, in order to improve the water resistance of the preparations still further.

The following examples are intended to clarify the present invention without limiting it. The numerical values in the examples signify percentages by weight, relative to the total weight of the respective preparations.

### EXAMPLES

### 1. Structural Modification

The structural modification is carried out by means of a ball mill or a ball mill operating continuously. The after-grinding is carried out by means of an air-jet mill, a toothed-disc mill or a pin-type mill.

TABLE 2
Preparation of the structurally modified titanium dioxides

| | | Structural | |
|---|---|---|---|
| | Oxide* | Modification | After-Grinding** |
| Example 1 | 1 | Yes | No |
| Example 2 | 1 | Yes | TM |
| Example 3 | 1 | Yes | AJ |
| Example 4 | 1 | Yes | AJ |
| Example 5 | 2 | Yes | No |
| Example 6 | 2 | Yes | TM |
| Example 7 | 2 | Yes | AJ |
| Example 8 | 2 | Yes | AJ |

*Oxide 1 = Aeroxide ® TiO₂ P 25
Oxide 2 = Aeroxide ® TiO₂ PF 2
**TM = toothed-disc mill
AJ = air-jet mill

TABLE 3
Physicochemical data of the structurally modified
titanium dioxides

| | | | | | Spec. |
|---|---|---|---|---|---|
| | | | | | Surface |
| | Tamped | | Drying | | Area acc. |
| | density | pH | Loss | Annealing | to BET |
| | [g/l] | Value | [%] | Loss [%] | [m²/g] |
| Example 1 | 805 | 3.6 | 1.2 | 1.1 | 53 |
| Example 2 | 735 | 3.6 | 1.0 | 0.7 | 52 |
| Example 3 | 324 | 3.8 | 0.7 | 1.1 | 52 |
| Example 4 | 446 | 3.8 | 0.6 | 1.2 | 50 |
| Example 5 | 860 | 3.7 | 1.4 | 1.8 | 58 |
| Example 6 | 764 | 3.6 | 1.4 | 1.3 | 58 |
| Example 7 | 351 | 3.8 | 1.2 | 1.2 | 56 |
| Example 8 | 299 | 3.8 | 1.1 | 1.3 | 59 |

### 2. Sunscreen Formulations

Preparation of Dispersion

278.25 g TEGOSOFT® TN are charged in a 500 ml PE cup, and 21.75 g of the titanium-dioxide powder to be investigated are stirred in with the aid of a dissolver (Pendraulik type LM34 No. 29490, disc diameter 6 cm) at 470 rpm and then dispersed for five minutes at 3000 rpm.

Subsequent to this, the dispersion is dispersed with an Ultra-Turrax stirrer (Polytron PT3100, dispersing tool PT-DA 3020/2 EC) for two minutes at 15,000 rpm.

Finally, the dispersion is dispersed for a further five minutes in a water-cooled container with the Ultra-Turrax stirrer at 15,000 rpm, use now being made of the dispersing tool PT-DA 3030-6060/3 EC.

UV-Vis Spectra (Transmission at 380 nm)

The UV-Vis spectra of 3 wt. % dispersions are measured in a detachable 10 μm quartz-glass cell with a UV-Vis spectrophotometer Specord 200 with photometer sphere (Analytik Jena AG). To this end, the oily dispersions described above are diluted with Tegosoft TN.

Subject to stirring with the dissolver (Pendraulik type LM34 No. 29490, disc diameter 5 cm; 1000-4000 rpm), AEROSIL® 200 is then added in portions, in order to produce a gel-like mass and to stabilise the oxide.

After the last addition of AEROSIL, redispersing has to be carried out for at least 2 minutes, in order to guarantee a homogeneous distribution of the AEROSIL. As a result, the transmission (%) is obtained within the range from 290 nm to 500 nm.

Viscosity or Thickening Effect

The viscosity is ascertained with a Brookfield Rheometer RVDV-III+cP. The measurement is taken in a PE mixing cup (350 ml) and in the RV spindle set, at 10 rpm. After 1 minute the value in mPa.s is read off.

TABLE 4
Characterisation of structurally modified
AEROXIDE ® TiO₂ P 25 samples

| | After- | Tamped | Transmission at | Viscosity (mPa · s) |
|---|---|---|---|---|
| Designation | grinding | density (g/l) | 380 nm (%) | (spindle type) |
| Example 1 | — | 805 | 34 | 80 (1) |
| Example 2 | TM | 735 | 45 | 63 (1) |
| Comparative | — | 282 | 22 | 520 (2) |
| Example | | | | |
| AEROXIDE | | | | |
| TiO₂ P25 | | | | |

Advantages of the products according to the invention from Examples 1 and 2 in comparison with the Comparative Example with AEROXIDE TiO₂ P 25 (not structurally modified) are:
A significantly increased tamped density. This means simplified handling and processing, less transportation and storage costs, and also reduced formation of dust.
A distinctly increased transmission at 380 nm. This means an increased transparency or, to be more precise, reduced whitening of sunscreen formulations prepared therewith.

A considerably reduced viscosity or thickening effect. This enables the preparation of dispersions or sunscreen formulations having a higher content of titanium dioxide and therefore having a higher protection factor.

Sunscreen Formulation 1

| | % | Constituent | |
|---|---|---|---|
| A. | 3.00 | Isopropyl myristate | Isopropyl myristate |
| | 8.00 | Jojoba oil | Simmondsia chinensis (jojoba) |
| | | | seed oil |
| | 4.00 | Uvinul ® MC 80 | Octyl methoxycinnamate |
| | 1.00 | Abil ® 350 | Dimethicone |
| | 6.00 | Cremophor ® WO 7 | PEG-7 hydrogenated castor oil |
| | 2.00 | Ganex ® V 216 | PVP/hexadecene copolymer |
| | 2.00 | Elfacos ® ST 9 | PEG-45/decyl glycol copolymer |
| | 2.00 | Uvinul ® MBC 95 | 4-methylbenzylidene camphor |
| B | 3.00 | Finely divided | Titanium dioxide |
| | | titanium dioxide | |
| | 5.00 | Z-Cote ® HP 1 | Zinc oxide (and) dimethicone |
| C | 1.00 | Magnesium | Magnesium sulfate |
| | | sulfate-7-hydrate | |
| | 5.00 | Glycerin 87% | Glycerin |
| | 0.20 | Edeta ® BD | Disodium EDTA |
| | 0.30 | Germoll ® 115 | Imidazolidinyl urea |
| | 57.00 | Water dem. | Water |
| | q.s. | Perfume | |
| | 0.50 | Euxyl ® K3000 | Phenoxyethanol, methyl |
| | | | paraben, butyl paraben, ethyl |
| | | | paraben, propyl paraben, |
| | | | isobutyl paraben |

Phase A is heated to 80° C., phase B is added, and homogenising is carried out for 3 minutes.

Phase C is heated to 80° C. and is stirred into the mixture of phases A and B, subject to homogenising.

Sunscreen Formulation 2

| | % | Constituent | |
|---|---|---|---|
| A. | 6.00 | Cremophor ® WO 7 | PEG-7 hydrogenated castor oil |
| | 2.00 | Elfacos ® ST 9 | PEG-45/dodecyl glycol |
| | | | copolymer |
| | 3.00 | Isopropyl | Isopropyl myristate |
| | | myristate | |
| | 8.00 | Jojoba oil | Jojoba (Buxus chinensis) oil |
| | 4.00 | Uvinul ® MC 80 | Octyl methoxycinnamate |
| | 2.00 | Uvinul ® MBC 95 | 4-methylbenzylidene camphor |
| | 3.00 | 1. Finely divided | Titanium dioxide |
| | | titanium dioxide | |
| | 1.00 | Abil ® 350 | Dimethicone |
| | 5.00 | Z-Cote ® HP 1 | Zinc oxide, dimethicone |
| B | 0.20 | Edeta ® BD | Disodium EDTA |
| | 5.00 | Glycerin 87% | Glycerin |
| | q.s. | Preserving agent | |
| | 60.80 | Water dem. | Aqua dem. |
| C | q.s. | Perfume | |

Phases A and B are heated separately to 85° C. Subsequently phase B is stirred into phase A and homogenised. The mixture is cooled to 40° C., phase C is added, and subsequently homogenising is again carried out.

Results

| | Formulation 1 | Formulation 2 |
|---|---|---|
| | Transparency | Transparency |
| | on the skin | on the skin |
| Comparative Example | unsatisfactory | unsatisfactory |
| AEROXIDE TiO₂ T805 | | |
| Example 2 | satisfactory | satisfactory |

1. A pyrogenically prepared, structurally modified titanium dioxides or pyrogenically prepared, structurally modified titanium-dioxide mixed oxides.

2. A process for preparing the pyrogenically prepared, structurally modified titanium dioxides or the pyrogenically prepared, structurally modified titanium-dioxide mixed oxides according to claim 1, comprising treating the pyrogenically prepared titanium dioxide or the pyrogenically prepared, structurally modified titanium-dioxide mixed oxide with a ball mill.

3. In a process for producing a sun screen formulation wherein the improvement comprises adding the pyrogenically prepared, structurally modified titanium dioxides or the pyrogenically prepared, structurally modified titanium-dioxide mixed oxides according to claim 1 to the sun screen formulary.

4. In a sunscreen formulation wherein the improvement comprises pyrogenically prepared, structurally modified titanium dioxides or pyrogenically prepared, structurally modified titanium-dioxide mixtures according to claim 1.

5. The process of claim 2 further comprising grinding the ball mill treated pyrogenically prepared titanium dioxide or the pyrogenically prepared, structurally modified titanium-dioxide mixed oxide.
